(19) United States
(12) Patent Application Publication
(10) Pub. No.: US 2004/0142968 A1
(43) Pub. Date: Jul. 22, 2004

(54) Method and kit for treating lower bowel pain

(76) Inventors: Donald Price, Gainesville, FL (US); G. Nicholas Verne, Gainesville, FL (US); Arup Sen, Gainesville, FL (US)

Correspondence Address: MARTIN L. MCGREGOR, MCGREGOR & ASSOCIATES, 26415 OAK RIDGE DRIVE, SPRING, TX, 77380, US

(21) Appl. No.: 10/347,048

(22) Filed: Jan. 17, 2003

(51) Int. Cl. A61K 31/445; A61K 31/24

(52) U.S. Cl. 514/317; 514/535

(57) ABSTRACT

Lower bowel pain such as that associated with menses, premenstrual syndrome (PMS) or irritable bowel syndrome is alleviated in a mammal suffering therefrom by administering to the mammal a pain alleviating amount of nontoxic local anesthetic such as lidocaine, lidocaine hydrochloride, bupivicaine, and the like. These local anesthetics will be applied as an injected intra rectal jelly, intra rectal suppository, or orally in capsule form.

### TECHNICAL FIELD

The invention is relevant to the field of pain management in a patient and specifically to the treatment of lower bowel pain especially pain associated with menses, premenstrual syndrome (PMS), irritable bowel syndrome (IBS) and other conditions.

### BACKGROUND OF THE INVENTION

This invention relates to methods for the specific treatment of pain that arises from nerve impulses from the colon or rectum and especially such pain associated with PMS, IBS and other functional bowel disorders in which there is chronic lower abdominal pain. An example is that of bowel pain associated with menses or pre-menses. Exclusions to this use include intra-rectal local anesthetics during conditions where there exists suspected or demonstrated organic pathology, such as appendicitis.

The applicants are not aware of any patents disclosing treatment of lower bowel pain associated with chronic non-pathological conditions by administration of a formulation of a local anesthetic that has not been subjected to conditions that lead to the formation of a complex of the anesthetic with a component of the formulation. The use of local anesthetics, preferably lidocaine, to treat ulcerative proctitis and colitis is disclosed in Ahlman et al., U.S. Pat. No. 5,331,013. In contrast to irritable bowel syndrome, the ulcerative conditions exhibit distinct lesions and may require surgical intervention if not successfully treated by non-invasive means.

In U.S. Pat. No. 6,071,959, Rhodes et al. disclose treatment of conditions including irritable bowel syndrome (IBS) with a complex formed of at least one amide-type anesthetic, such as lidocaine, and a polyacrylate. Rhodes states that lignocaine has an anaesthetic effect of only intermediate duration. The duration of the topical anaesthetic may be prolonged through being present in the form of a polyacrylate complex. In contrast, no complexing agent was used in the present invention and excellent results were obtained. U.S. Pat. No. 5,785,994 and several related patents, disclose an osmotic release device used to administer a large group of medications including local anesthetics to the intestines to treat a long list of disorders including IBS, but does not specifically teach the treatment of IBS by the local administration of anesthetics.

The IBS is one of the most common disorders seen by gastroenterologists. Patients classically present with chronic abdominal pain associated with an alteration in bowel habits. It is now well accepted that the majority of patients with IBS demonstrate enhanced perception of balloon distension of the rectum. This visceral hypersensitivity is manifested by increased intensity of sensations, lowered thresholds for visceral pain, and/or exaggerated viscerosomatic referral in comparison to control subjects (Mertz et al., 1995; Naliboff et al; 1997; Verne et al., 2001a). The diagnosis of IBS is based on the ROME II criteria and exclusion of organic disease (Thompson et al., 1999). These criteria include: 1) At least 12 weeks of abdominal pain that need not be consecutive in the preceding 12 months; 2) Abdominal discomfort or pain that has 2 or more of 3 features-relieved with defecation, onset associated with a change in frequency of stool, onset associated with a change in form (appearance of stool).

There are very few approaches to treating pain in IBS, anal fissures, internal/external hemorrhoids, rectal prolapse, proctalgia fugax, levator ani syndrome, and pudendal canal syndrome. The only current FDA approved treatment is for the constipation associated with IBS (Tegaserod maleate). Surprisingly, it has now been found that the non-toxic local anesthetic lidocaine exhibits a strong pain ameliorating effect in IBS patients that lasts at least 4 hours. Moreover, it also has been found that this same dose of lidocaine jelly potently reduces lower bowel pain associated with menses.

### SUMMARY OF THE INVENTION

In accordance with the present invention, there is provided a method specifically for the treatment of lower bowel pain such as that associated with IBS and menses, that comprises administering to a mammal exhibiting lower bowel or rectal pain a pain-relieving amount of at least one nontoxic local anesthetic, preferably acrylate free, and preferably in jelly or capsule form. This method of treating pain associated with IBS and other chronic pain disoders of the lower bowel is heretofore unrecognized and not practiced in medicine. The method of administration includes mainly direct intrarectal methods such as suppositories or applicators similar to those used in vaginal delivery of medications and local injections of lidocaine jelly via syringes. Alternatively, for females intra-vaginal application may be used. The prefered methods include oral administration by use of capsules that release the dose in the lower bowel such as timed release and pH sensitive capsules to enable the release of the anesthetic when the capsule reaches the lower bowel. The invention provides a method of treating pain associated with pre-menses or menses that comprises administering in the rectum, colon or vagina of a human female exhibiting pain associated with menses or pre-menses a pain-alleviating amount of at least one non-toxic local anesthetic.

More specifically the invention provides method of reducing pain that arises from nerve impulses from the colon or rectum and is associated with a condition selected from the group consisting of menses, premenstrual syndrome, irritable bowel syndrome, anal fissures, internal/external hemorrhoids, rectal prolapse, proctalgia fugax, levator ani syndrome, and pudendal canal syndrome that comprises administering in the colon of a mammal exhibiting pain associated with irritable bowel syndrome, anal fissures, internal/external hemorrhoids, rectal prolapse, proctalgia fugax, levator ani syndrome, and pudendal canal syndrome by the application of a pain-alleviating amount of at least one non-toxic local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride. Preferably the non-toxic local anesthetic is selected from the group consisting of lidocaine, lidocaine hydrochoride, bupivivaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride. More preferably the local anesthetic is provided as an intrarectal suppository with 2% lidocaine jelly as the principle ingredient. In another preferred embodiment the local anesthetic is provided as the nontoxic local anesthetic is provided as an intrarectal jelly administered by a plastic syringe or plastic rectal tube. In a further preferred embodiment the local anesthetic is provided by intrarectal sustained release capsules. Alternatively the local anesthetic may be provided by oral administration and is selectively released in the colon. A preferred embodiment provides oral sustained release capsules, more preferably capsules that dissolve at a pH<6.0 and are selectively released in the colon.

A further embodiment provides a kit specially adapted to practice the invention. A preferred embodiment provides a kit for treating lower bowel pain that comprises an applicator, a dose of a local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride and an instruction sheet describing a treatment for pain associated with a condition selected from the group consisting of menses, premenstrual syndrome, irritable bowel syndrome, anal fissures, internal/external hemorrhoids, rectal prolapse, proctalgia fugax, levator ani syndrome, and pudendal canal syndrome. A preferred kit provides a dose of 2% lidocaine jelly. Another preferred kit provides a sustained release dose.

Alternate emboidiments of the invention include a method of treating lower bowel pain that comprises administering in the rectum or colon of a human exhibiting pain associated with irritable bowel syndrome a pain-alleviating amount of at least one non-toxic local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride.

Another embodiment provides a method of reducing pain that arises from nerve impulses from the colon or rectum and is associated with a chronic condition that comprises administering in the colon of a mammal exhibiting lower bowel pain a pain-alleviating amount of at least one non-toxic local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride.

Alternatively the invention provides a method of reducing pain that arises from nerve impulses from the colon or rectum and is associated with a non ulcerative chronic condition that comprises administering in the colon of a mammal exhibiting pain from nerve impluses from the rectum or lower bowel, a pain-alleviating amount of at least one non-toxic polyacrylate free local anesthetic.

### BRIEF DESCRIPTION OF THE DRAWINGS

FIG. 1 is a graphical presentation of test results showing the therapeutic effects of 300 mg intrarectal lidocaine jelly as compared to intrarectal placebo saline jelly in 10 patients with pain associated with IBS. These agents were administered on a double-blind basis.

FIG. 2 is a graphical presentation of the test results shown in FIG. 1 plotted as pain intensity difference scores (PID). PID was calculated by averaging the baseline rating of each patient and then subtracting the post-treatment pain intensity rating at a given time point from average baseline rating. Thus, there were 7 PID scores for both the lidocaine jelly and placebo condition. Each data point is the mean PID score and standard error of the 10 patients.

FIG. 3 is a graphical presentation of test results showing the therapeutic effects of 300 mg intrarectal lidocaine jelly as compared to intrarectal placebo saline jelly in a patient with bowel pain associated with menses. These agents were administered on a double-blind basis.

### DETAILED DESCRIPTION OF THE INVENTION

IBS pain is a common variety of visceral pain and it may occur as least partly as a result of abnormal functioning of the peripheral and/or central nervous system. A critical component of this condition may be the existence of tonic impulse input from peripheral nerves supplying the rectum and/or lower colon (Verne et al., 2001)

Although it has been shown that that experimentally induced rectal pain can be reduced by intrarectal lidocaine jelly (Verne et al., 2002, submitted for publication in Pain now in revision), it is not presently known whether lidocaine jelly has a direct pain-reducing effect on clinical pain naturally experienced by IBS patients. Rectal distension delivered by a barostat balloon does not constitute a form of clinical pain and in fact is not used as a clinical test by gastroenterologists or other physicians. IBS patients have varied symptomatology that may include both abdominal and rectal pain. Since the area of gastrointestinal abnormality may not be restricted to the rectum, it is unclear from the study of Verne et al. (Pain, in revision, 2002) whether application of rectal lidocaine jelly would have effects on spontaneous ongoing pain in IBS patients. A more complete demonstration of the therapeutic benefits of such an approach requires a study in which ongoing clinical pain is assessed for a longer period of time in IBS patients. Thus, we conducted a study to determine whether administration of 300 mg lidocaine in a jelly form could ameliorate the ongoing clinical pain associated with irritable bowel syndrome.

Another form of bowel pain that may be treated with lidocaine jelly or other local anesthetic preparations is that of Premenstrual syndrome (PMS). PMS is a common gynecological condition characterized by severe abdominal/pelvic pain prior to menses. In addition, patients with this condition often complain of alterations in their bowel patterns (i.e. constipation/diarrhea). Currently, there are few medical therapies available for PMS. Currently, aspirin products and hormonal therapy are used to treat PMS with little efficacy and the risk of side effects.

### Definitions

The term “nontoxic” as used herein shall be understood in a relative sense and is intended to designate any local anesthetic substance that has been approved by the United States Food and Drug Administration (“FDA”) or an equivalaent regulatory authority in a major Western country for administration to humans or, in keeping with established criteria, is likely to be deemed appropriate for administration to humans. The concept of “nontoxic” as used herein also shall be understood to be interrelated with the method of administration. Thus, whereas intrarectal liquid lidocaine has been shown to result in detectable and even pharmacologically significant blood levels of lidocaine (de Boer et al., 1979), intrarectal lidocaine jelly administration has been shown to not result in detectable blood levels of lidocaine. Blood lidocaine levels >5.0 mg/L are toxic. That lidocaine jelly is safe is further attested by the fact that barostat catheters used for endoscopy are commonly coated with lidocaine jelly with amounts of lidocaine at 300 mg or greater. However, the purpose of using lidocaine jelly during endoscopy is not that of reversing pain of IBS, PMS or other chronic conditions.

The term “polyacrylate free” means a material that contains essentially no polyacrylate. The term “uncomplexed” means a preparation in a formulation of a non-toxic local anesthetic that has not been exposed to a condition that is likely to result in a complex that is distinct from the anesthetic based on physical or chemical analytical methods.

### General Description

Among the nontoxic agents that are useful as local anesthetics in practicing the claimed method are lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, tetracaine hydrochloride, and the pharmaceutically acceptable salts thereof.

Administration of the nontoxic local anesthetic, such as lidocaine, can be by way of rectal or oral administration. In the case of females, vaginal adminstration may also be used. Effective dosage levels can vary widely, e.g., from 50 to 1200 mg during a twenty four hour period, but actual amounts will, of course, depend on the state and circumstances of the patient being treated. As those skilled in the art recognize, many factors that modify the action of the active substance herein will be taken into account by the treating physician such as the age, body weight, sex, diet and condition of the patient, the time of administration, the rate and route of administration, and so forth. Optimal doses for a given set of conditions can be ascertained by those skilled in the art using conventional dosage determination tests in view of the experimental data provided herein.

The nontoxic local anesthetic will ordinarily be formulated with one or more pharmaceutically acceptable ingredients in accordance with known and established practice. Thus, the local anesthetic can be formulated as an injectable jelly, suppository, powder, etc. Formulations for oral use can be provided as hard gelatin capsules that are pH sensitive and release their contents into the lower bowel or rectum. The capsules may also be sustained release capsules that release their contents in the lower bowel. Formulations for oral use may include mixing the local anesthetic with an inert diluent such as calcium carbonate, calcium phosphate or kaolin, or as soft gelatin capsules wherein the local anesthetic is mixed with an oleaginous medium, e.g., liquid paraffin or olive oil.

A typical useful formulation of the invention includes from 0.5 to 5% local anesthetic, one or more preservatives such as methylparaben or propylparaben, one or more carriers such as hydroxypropylmethylcelluslose and pH adjusting materials such as an acid, for example hydrocholric acid, a base, for example sodium hydroxide to provide a final pH in the range of 6.0 to 7.0. In general any usual formulation of a pharmacuetically acceptable blend of ingredients that delivers a dose of local anesthetic material in a nontoxic, preferably uncomplexed state to the lower bowel is suitable for use in the method and kit of the invention.

### EXAMPLES THAT ARE ILLUSTRATIVE OF THE INVENTION

### IBS Subjects

All patients gave written informed consent to participate in the study. Patients with IBS as diagnosed by Dr. Nicholas Verne were recruited to participate. Ten Caucasian pre-menopausal women with IBS participated in the study. The mean age was 33 (±4) years. All participants completed a GI symptom questionnaire and received a physical examination by an experienced gastroenterologist. The diagnosis of IBS was made based on the ROME II criteria and exclusion of organic disease (Thompson et al., 1999). All the patients had diarrhea-predominant IBS and had symptoms for at least 5 years. All of the IBS patients were carefully screened to verify that each patient had no clinical criteria for fibromyalgia or chronic pain condition.

All subjects were asked to fast for 12 hours and self-administer one Fleets® enema (CB Fleet Co., Inc., Lynchberg, Va.) at least two hours prior to arriving for the experimental procedures. None of the participants in this study were receiving pain medications, selective serotonin reuptake inhibitors, monoamine oxidase inhibitors, or tricyclic antidepressants prior to or during the course of the study. At the first testing session, patients were randomly assigned to receive either 300 mg lidocaine in a jelly form or saline jelly in random order that was counterbalanced across subjects. Patients were then tested in a second session 10 days later and were given the agent that they did not receive during the first session. Thus, the design was a crossover in which each patient was tested with both lidocaine and saline jelly. Both the patient and the examiner who administered the pain rating scales and the lidocaine/saline agents were blinded to the agent being administered during each session. A validated visual analogue scale was used to measure the subjects' perceived pain (Price, 1994).

### Protocol

The patients were greeted in a waiting room, escorted to an examination room, and introduced to the study. Prior to the study, patients signed an informed consent form stating that they would receive either an active analgesic or a placebo agent during the treatment sessions. Each patient's ratings of their ongoing rectal pain were obtained under three conditions, including Natural History (NH) Rectal Lidocaine (RL), Rectal Placebo (RP). A one-hour NH condition preceded either the RL or RP administration in both sessions. Thus, patients rated their rectal pain for one hour at 15-minute intervals before injection of the rectal agent (NH) and 15 min, 30 min, 45 min, 1 hour, 2 hours, 4 hours, and 6 hours afterwards. Both agents were applied through a plastic syringe. Neither agent contained polyacrylate, and the lidocaine was uncomplexed. After all of the sessions were completed, the patients were debriefed.

### Data Analysis

The four baseline ratings were averaged for each patient to create a single baseline score for both placebo and lidocaine conditions. The pain rating at each post-treatment time point was subtracted from the baseline rating to create a pain intensity difference score (PID) for each post-treatment time point of each patient. The sum of these PIDs across all post-treatment time points, termed SPID, was then used as a repeated measure in a two-tailed paired t-test that compared placebo and lidocaine conditions. This approach is well established in the literature on measuring outcomes of analgesic treatments and is considered an adequate measure of clinical analgesic efficacy (Farrar et al., 2000). After demonstrating statistical significance between SPID scores, paired t tests were then used to determine which time points exhibited significant differences in mean PID scores. An alpha of 0.02 was chosen.

### Non IBS patient

A 58 year-old Caucasian female patient on estrogen supplementation having lower bowel pain associated with menses was tested according to the protocol described above for IBS patients. An N-of-one design was used and pain ratings following rectal lidocaine and rectal placebo treatments were compared using a two tailed t-test.

### EXAMPLE 1

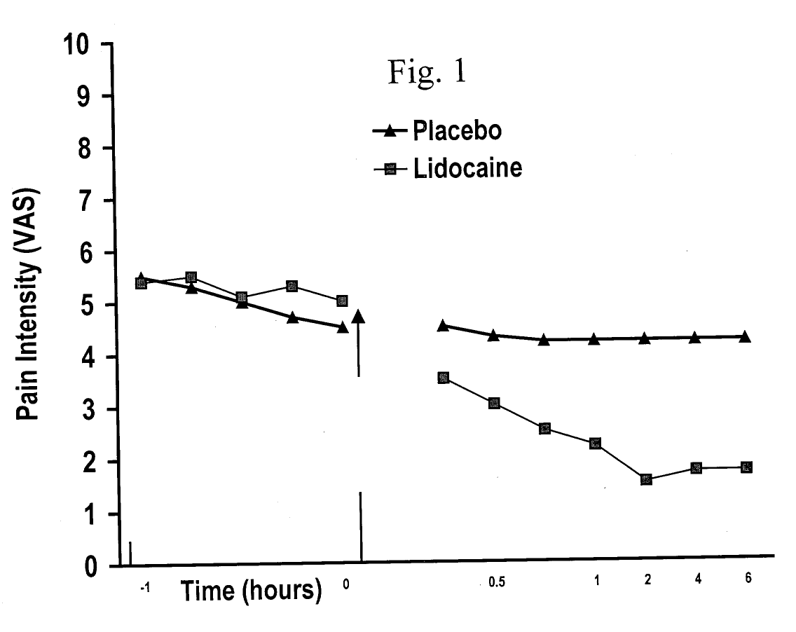

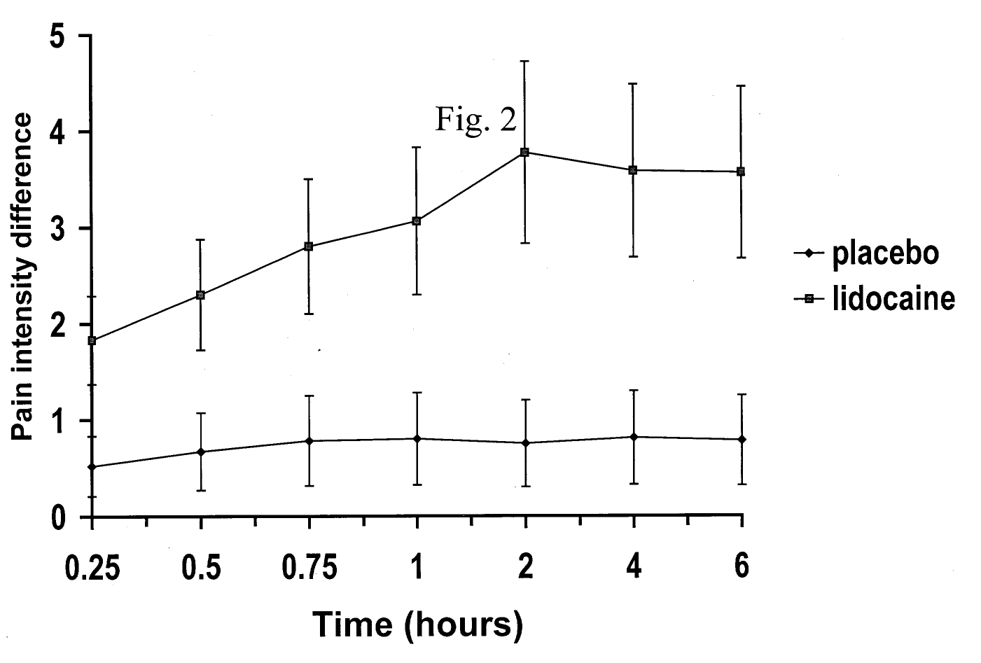

Reduction of clinical IBS pain by intrarectal lidocaine jelly. FIGS. 1 and 2 illustrate mean pain ratings and mean pain intensity difference scores respectively. This example demonstrates the effectiveness of intrarectal lidocaine jelly in reducing pain associated with IBS. When comparing post-treatment conditions, the mean SPID score of the lidocaine condition was significantly greater than that of the placebo condition (t=3.16; P<0.02). PID (average pain intensity difference) scores were significantly different between lidocaine and placebo conditions at the following time points shown in FIG. 2: 30 minutes (P<0.02), 45 minutes (P<0.01), 1 hour (P<0.001), 2 hours (P<0.01), 4 hours (P<0.02) (FIG. 2). Differences between mean PID scores during placebo and lidocaine conditions were not statistically significant at 15 minutes and 6 hours (both P>0.02).

### EXAMPLE 2

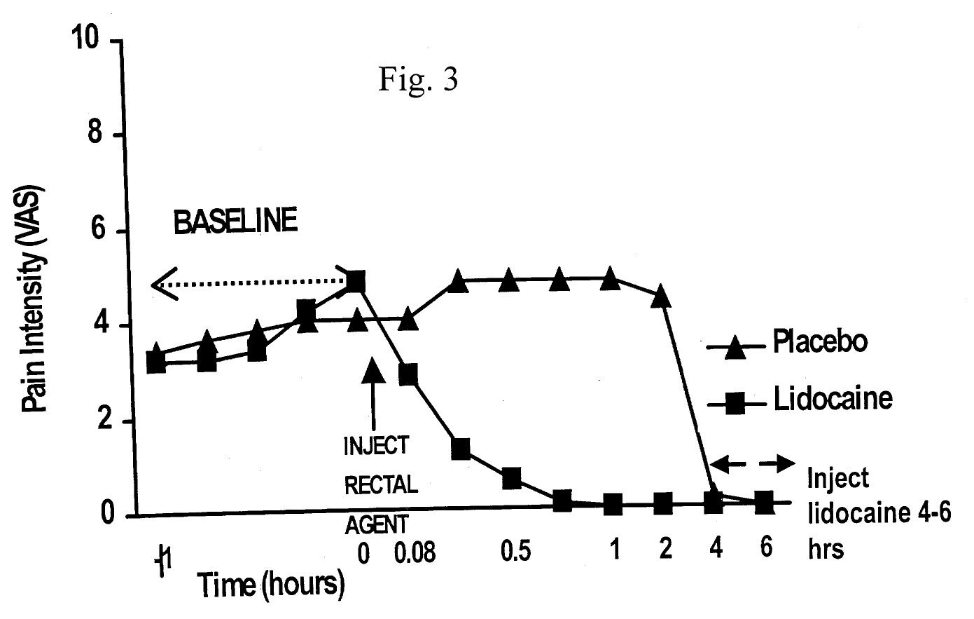

Reduction of clinical pain in a patient with bowel pain associated with menses. Intrarectal lidocaine jelly and saline jelly were applied according to the same double blind protocol as described above for IBS patients. In a N-of-one design, using a t test to compare the 6 time points following each treatment, the mean pain intensity during the lidocaine treatment was significantly lower than during the placebo saline treatment (P<0.02). The results are shown in FIG. 3. Note the complete reversal of pain after lidocaine but not saline jelly. An open injection of lidocaine at 4 hours after the placebo saline administration also reversed bowel pain associated with pre-menses.

We claim:

1. A method of reducing pain that arises from nerve impulses from the colon or rectum and is associated with a condition selected from the group consisting of menses, premenstrual syndrome, irritable bowel syndrome, anal fissures, internal/external hemorrhoids, rectal prolapse, proctalgia fugax, levator ani syndrome, and pudendal canal syndrome that comprises administering in the colon of a mammal exhibiting pain associated with irritable bowel syndrome, anal fissures, internal/external hemorrhoids, rectal prolapse, proctalgia fugax, levator ani syndrome, and pudendal canal syndrome by the application of a pain-alleviating amount of at least one non-toxic local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride.

2. The method of claim 1 wherein the nontoxic local anesthetic is provided as an intrarectal suppository with 2% lidocaine jelly as the principle ingredient.

3. The method of claim 1 wherein the nontoxic local anesthetic is provided as an intrarectal jelly administered by a plastic syringe or plastic rectal tube.

4. The method of claim 1 wherein the nontoxic local anesthetic is provided by intrarectal sustained release capsules.

5. The method of claim 1 wherein the nontoxic local anesthetic is provided by oral administration and is selectively released in the colon.

6. The method of claim 1 wherein the nontoxic local anesthetic is provided by oral sustained release capsules that dissolve at a pH<6.0 and is selectively released in the colon.

7. The method of claim 1 wherein the dose is polyacrylate free.

8. A method of treating pain associated with pre-menses or menses that comprises administering in the rectum, colon or vagina of a human female exhibiting pain associated with menses or pre-menses a pain-alleviating amount of at least one non-toxic local anesthetic.

9. The method of claim 8 wherein the non-toxic local anesthetic is selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride.

10. The method of claims 8 wherein the nontoxic local anesthetic is provided as an intra-rectal or vaginal suppository with 2% lidocaine jelly as the principle ingredient.

11. The method of claim 8 wherein the nontoxic local anesthetic is provided as an intra-rectal or vaginal jelly administered by a plastic syringe or plastic rectal tube

12. The method of claim 8 wherein the nontoxic local anesthetic is provided by intra rectal sustained release capsules.

13. The method of claim 8 wherein the nontoxic local anesthetic is provided by oral administration and is selectively released in the colon.

14. The method of claim 8 wherein the nontoxic local anesthetic is provided by oral sustained release capsules that dissolve at a pH<6.0 and is selectively released in the colon

15. A kit for treating lower bowel pain that comprises an applicator, a dose of a local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride and an instruction sheet describing a treatment for pain associated with a condition selected from the group consisting of menses, premenstrual syndrome, irritable bowel syndrome, anal fissures, internal/external hemorrhoids, rectal prolapse, proctalgia fugax, levator ani syndrome, and pudendal canal syndrome.

16. The kit of claim 15 wherein the dose comprises 2% lidocaine jelly as the principle ingredient.

17. The kit of claim 15 wherein the dose is provided in a sustained release form.

18. A method of treating lower bowel pain that comprises administering in the rectum or colon of a human exhibiting pain associated with irritable bowel syndrome a pain-alleviating amount of at least one non-toxic local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride.

19. The method of claim 18 wherein the nontoxic local anesthetic is provided as an intra-rectal suppository with 2% lidocaine jelly as the principle ingredient.

20. The method of claim 18 wherein the nontoxic local anesthetic is provided as an intra-rectal jelly administered by a plastic syringe or plastic rectal tube

21. The method of claim 18 wherein the nontoxic local anesthetic is provided by intra rectal sustained release capsules.

22. The method of claim 18 wherein the nontoxic local anesthetic is provided by oral administration and is selectively released in the colon

23. The method of claim 18 wherein the nontoxic local anesthetic is provided by oral sustained release capsules that dissolve at a pH<6.0 and is selectively released in the colon

24. A method of reducing pain that arises from nerve impulses from the colon or rectum and is associated with a chronic condition that comprises administering in the colon of a mammal exhibiting lower bowel pain a pain-alleviating amount of at least one non-toxic local anesthetic selected from the group consisting of lidocaine, lidocaine hydrochoride, bupivicaine hydrochloride, levobupivicaine ((S)-1-butyl-N-(2,6-dimethylphenyl)-2-piperidinecarboxyamide), chloroprocaine hydrochloride, dibucaine, dibucaine hydrochloride, etidocaine hydrochloride, mepivicaine hydrochloride, piperocaine hydrochloride, prilocaine hydrochloride, procaine hydrochloride, propoxycaine hydrochloride, tetracaine, and tetracaine hydrochloride.

25. The method of claim 24 wherein the nontoxic local anesthetic is provided as an intra-rectal suppository with 2% lidocaine jelly as the principle ingredient.

26. The method of claim 24 wherein the nontoxic local anesthetic is provided as an intra-rectal jelly administered by a plastic syringe or plastic rectal tube

27. The method of claim 24 wherein the nontoxic local anesthetic is provided by intra rectal sustained release capsules.

28. The method of claim 24 wherein the nontoxic local anesthetic is provided by oral administration and is selectively released in the colon

29. The method of claim 24 wherein the nontoxic local anesthetic is provided by oral sustained release capsules that dissolve at a pH<6.0 and is selectively released in the colon

30. A method of reducing pain that arises from nerve impulses from the colon or rectum and is associated with a chronic condition that comprises administering in the colon of a mammal exhibiting tonic nerve input-caused lower bowel pain a pain-alleviating amount of at least one non-toxic polyacrylate free local anesthetic.

31. The method of claim 30 wherein the nontoxic local anesthetic is provided as an intra-rectal suppository with 2% lidocaine jelly as the principle ingredient.

32. The method of claim 30 wherein the nontoxic local anesthetic is provided as an intra-rectal jelly administered by a plastic syringe or plastic rectal tube

33. The method of claim 30 wherein the nontoxic local anesthetic is provided by intra rectal sustained release capsules.

34. The method of claim 30 wherein the nontoxic local anesthetic is provided by oral administration and is selectively released in the colon

35. The method of claim 30 wherein the nontoxic local anesthetic is provided by oral sustained release capsules that dissolve at a pH<6.0 and is selectively released in the colon
